(19) United States
(12) Patent Application Publication
(10) Pub. No.: US 2020/0037522 A1
(43) Pub. Date: Feb. 6, 2020

(54) ROBOTIC WATERING DEVICE FOR MAINTAINING LIVE PLANTS

(71) Applicant: Walmart Apollo, LLC, Bentonville, AR (US)

(72) Inventors: Timothy Ryan DeJarnette, Fayetteville, AR (US); Nicholas Hoyne, Rogers, AR (US); Kevin Reed, Bentonville, AR (US)

(21) Appl. No.: 16/165,898

(22) Filed: Oct. 19, 2018

Related U.S. Application Data
(60) Provisional application No. 62/712,795, filed on Jul. 31, 2018.

(51) Int. Cl. A01G 27/00; B25J 11/00; B25J 9/16

(52) U.S. Cl. CPC A01G 27/003; G01N 33/0098; B25J 9/1679; B25J 11/008

(57) ABSTRACT

Examples provide a robotic plant-watering device including a set of articulated robotic arms connected to a main body. One or more adjustable water sprinkler devices attach to one or more of the robotic arms for watering one or more selected plants. One or more gripper devices removably attach to one or more of the robotic arms to grip a portion of a plant or plant container. The gripper device is utilized to modify a plant's position or location. A set of sensor devices generate sensor data associated with the plants or the conditions within a live plant center. A plant maintenance component analyzes the sensor data using a set of plant maintenance rules to generate a dynamic plant watering schedules based on the plant status and ambient conditions. The plant-watering device autonomously sprays a predetermined amount of water specified in the dynamic plant watering schedule onto a selected plant.

### BACKGROUND

In a garden center or other plant nursery, there are frequently dozens or even hundreds of different types of plants to be maintained. Each type of plant frequently requires varying amounts of sun exposure, soil moisture levels, soil acidity (pH), watering frequency, fertilizer requirements and/or soil composition for maintenance, health and growth of the plants. Managing these diverse maintenance requirements for every type of plant on an individual basis is typically too time consuming, cost prohibitive and impractical in large-scale garden centers. In some solutions, plants are watered simultaneously/uniformly by water sprinklers spread throughout the garden center. This automated watering regime may lead to overwatering of some plants and underwatering of others, resulting in sub-optimal condition of plants and inefficient resource usage in plant maintenance.

### SUMMARY

Some examples provide a system for robotic plant watering based on dynamic context data. The system includes a set of sensor devices generating sensor data associated with a plurality of plants in a live plant center. The sensor data includes temperature data and/or image data. A plant maintenance component generates a dynamic per-plant watering schedule for one or more plants based on an analysis of the sensor data and real-time context data associated with the live plant center using a set of per-plant maintenance rules. A robotic plant-watering device waters one or more plants in the live plant center in accordance with the dynamic per-plant watering schedule. The robotic plant-watering device includes a set of water lines connecting a water tank to a set of articulating robotic arms. An adjustable watering apparatus attaches to a robotic arm. The watering apparatus releases a predetermined quantity of water from the water tank onto the one or more plants. A gripper device attaches to a robotic arm for grasping a plant or plant container. The gripper device adjusts the position or location of the plant from a first location to a second location. A control device includes a processor communicatively coupled to a memory. A controller component triggers release of a quantity of water from the watering apparatus onto one or more plants for a duration of time specified in the dynamic per-plant watering schedule.

Other examples provide a computer-implemented method for dynamically watering plants via a robotic plant-watering device. An analysis component analyzes sensor data generated by one or more sensor devices associated with plants in a live plant center and real-time context data associated with the live plant center using a set of status criteria. The analysis component generates an updated status for each type of plant in the live plant center based on a result of the analysis. A plant maintenance component updates a set of watering instructions for a selected plant type. The updated set of watering instructions includes a next scheduled watering time and an amount of water to be applied to a set of plants of the selected plant type during the next scheduled watering time. A navigation system generates a set of navigation instructions for navigating the robotic plant-watering device to a current location of the set of plants of the selected plant type. A control device triggers a robotic arm to release a predetermined quantity of water onto the selected plant for a predetermined duration of time in accordance with the updated set of watering instructions. The plant maintenance component updates historical watering data for the selected plant on a data storage to reflect completion of a watering task associated with the selected plant.

Still other examples provide a robotic plant-watering device including a set of articulated robotic arms connected to a main body. The set of robotic arms includes a set of adjustable water sprinkler devices removably attached to one or more articulated robotic arms. An adjustable water sprinkler device releases a quantity of water from a water source via a set of apertures on each water sprinkler device. The set of water sprinkler devices are adjustable to control the flow of water from the watering device and/or adjustable to control the direction of water flowing out of the watering device.

A set of sensor devices generate sensor data. The set of sensor devices includes a set of image capture devices and/or a set of water quality sensors associated with the water source. A control device includes a memory communicatively coupled to at least one processor. A plant maintenance component analyzes the sensor data using a set of plant maintenance rules to generate a dynamic plant watering schedules for a selected plant based on a current status of the selected plant and current ambient conditions within the plant center. A controller component moves at least one robotic arm within a proximity of the selected plant. The controller component triggers release of a quantity of water specified in the dynamic plant watering schedule for the selected plant onto the selected plant.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an exemplary block diagram illustrating a system for watering plants via a robotic plant-watering device based on dynamic context-data.

FIG. 2 is an exemplary block diagram illustrating a live plant center including at least one robotic plant-watering device.

FIG. 3 is an exemplary block diagram illustrating a robotic plant-watering device.

FIG. 4 is an exemplary block diagram illustrating a smart plant display storing a set of plants scheduled for watering.

FIG. 5 is an exemplary block diagram illustrating a robotic plant-watering device watering plants on a plant display.

FIG. 6 is an exemplary block diagram illustrating a robotic plant-watering device utilizing a set of gripper devices to re-arrange plants on a plant display.

FIG. 7 is an exemplary block diagram illustrating a set of plant displays including a set of plants re-arranged by a robotic plant-watering device.

FIG. 8 is an exemplary block diagram illustrating a robotic plant-watering device.

FIG. 9 is an exemplary block diagram illustrating an articulating robotic arm including an adjustable watering apparatus.

FIG. 10 is an exemplary block diagram illustrating an articulating robotic arm including a gripper device.

FIG. 11 is an exemplary block diagram illustrating an adjustable watering apparatus.

FIG. 12 is an exemplary block diagram illustrating a telescoping robotic arm for watering hanging plants.

FIG. 13 is an exemplary block diagram illustrating a robotic plant-watering device connecting to a water refill docking device.

FIG. 14 is an exemplary block diagram illustrating a set of sensor devices.

FIG. 15 is an exemplary block diagram illustrating a plant maintenance component.

FIG. 16 is an exemplary block diagram illustrating a cloud server including an analysis component.

FIG. 17 is an exemplary block diagram illustrating a database for storing plant maintenance data.

FIG. 18 is an exemplary flow chart illustrating operation of the computing device to generate instructions for a robotic plant-watering device.

FIG. 19 is an exemplary flow chart illustrating operation of the computing device to autonomously water plants.

FIG. 20 is an exemplary flow chart illustrating operation of the computing device to autonomously refill a water tank on a robotic plant-watering device.

Corresponding reference characters indicate corresponding parts throughout the drawings.

### DETAILED DESCRIPTION

A more detailed understanding may be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that may in isolation and out of context be read as absolute and therefore limiting, may only properly be read as being constructively preceded by a clause such as “In at least some embodiments, . . . ” For brevity and clarity of presentation, this implied leading clause is not repeated ad nauseum.

Referring to the figures, examples of the disclosure enable watering plants via a robotic plant-watering device based on real-time data associated with the plants and/or conditions within a live plant center. In some examples, the robotic plant-watering device autonomously moves into watering range of a plant and extends an articulating robotic arm including a watering apparatus near the plant. The robotic plant-watering device sprays water into the plant's container or onto a water absorbent mat under the plant. When the device finishes watering the plant, the robotic plant-watering device automatically repositions the robotic arm within range of a next plant to be watered and proceeds with watering the plant in accordance with dynamic plant watering instructions. This enables more efficient plant watering while improving quality of plant maintenance.

In other examples, the robotic plant-watering device includes a gripper device for grabbing and manipulating plants on a display. The device moves plants from one display area to another and/or repositions plants on the display to improve the arrangements/appearance of plants on the displays in the plant center. For example, the device may move plants to a warmer/protected area when there is a danger of frost, remove damaged, wilted or out-of-season plants, pick up plants that have fallen or tipped over, re-arrange plants which have been misplaced on an incorrect display, etc. The system provides the robotic device with instructions to water and/or move plants based on dynamic sensor data and context data for the plant center. This improves utilization of plant watering resources, improves efficiency of plant maintenance, corrects misplacements of plants, improves display appearance, and reduces water usage.

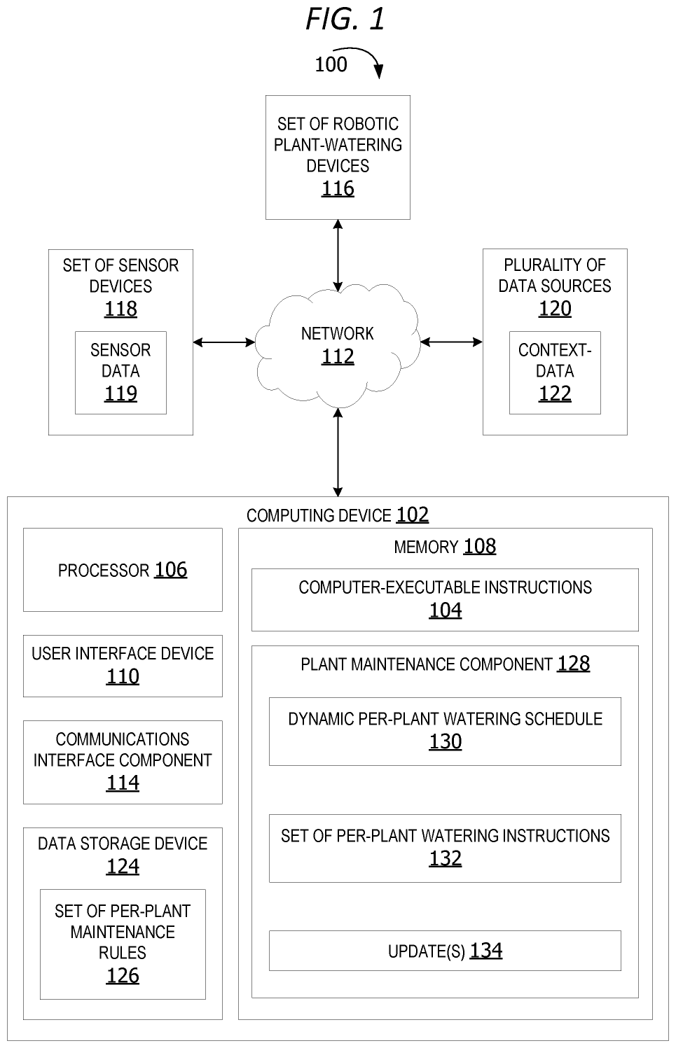

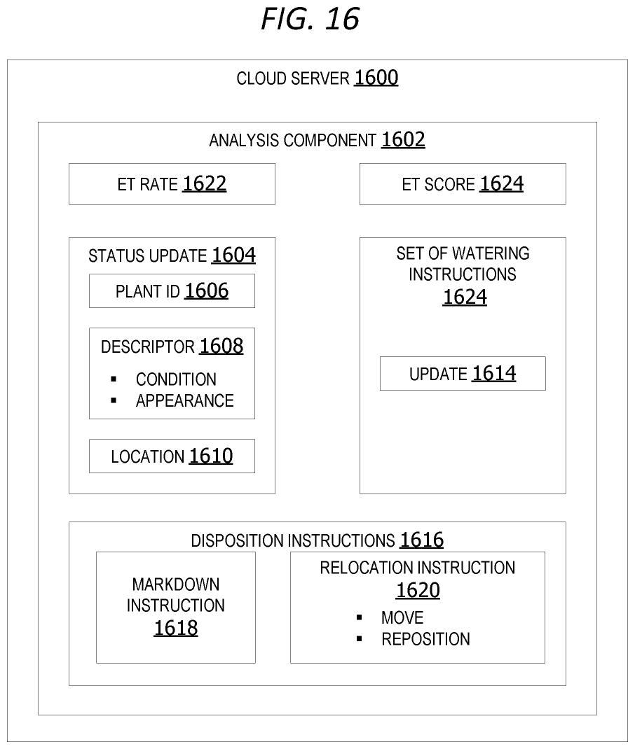

Referring again to FIG. 1, an exemplary block diagram illustrates a system 100 for watering plants via a robotic plant-watering device based on dynamic context-data. In the example of FIG. 1, the computing device 102 represents any device executing computer-executable instructions 104 (e.g., as application programs, operating system functionality, or both) to implement the operations and functionality associated with the computing device 102. The computing device 102 may include a mobile computing device or any other portable device. In some examples, the mobile computing device includes a mobile telephone, laptop, tablet, computing pad, netbook, gaming device, and/or portable media player. The computing device 102 may also include less portable devices such as servers, desktop personal computers, kiosks, or tabletop devices. The computing device 102 in other examples may be implemented on a cloud server, such as the cloud server 1600 in FIG. 16. Additionally, the computing device 102 may represent a group of processing units or other computing devices.

In some examples, the computing device 102 has at least one processor 106 and a memory 108. The computing device 102 may also include a user interface device 110.

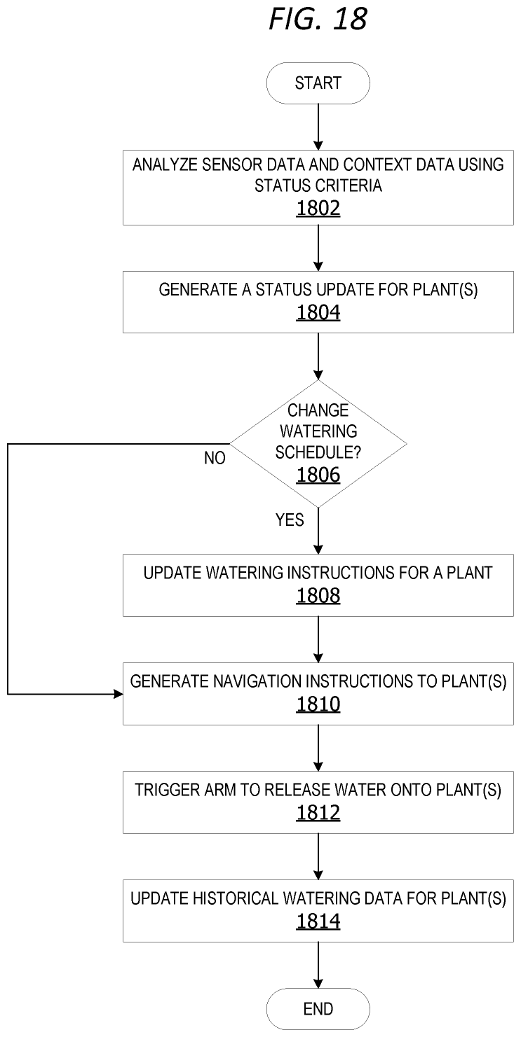

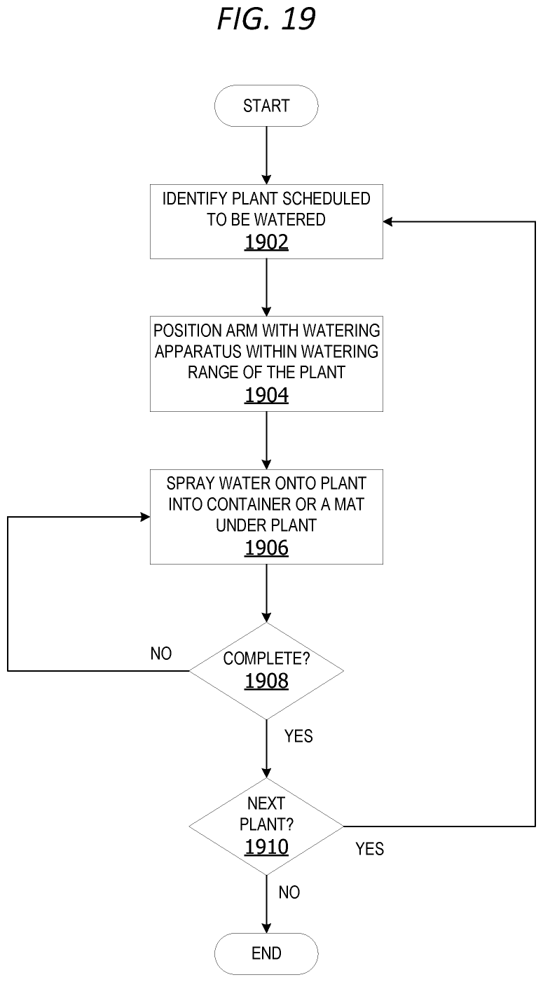

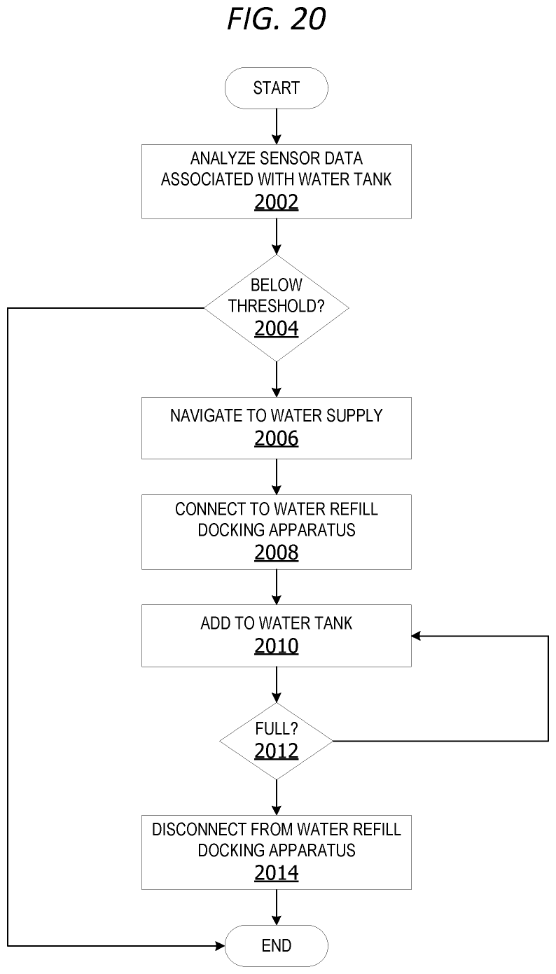

The processor 106 includes any quantity of processing units and is programmed to execute the computer-executable instructions 104. The computer-executable instructions 104 may be performed by the processor 106 or by multiple processors within the computing device 102 or performed by a processor external to the computing device 102. In some examples, the processor 106 is programmed to execute instructions such as those illustrated in the figures (e.g., FIG. 18, FIG. 19 and FIG. 20).

The computing device 102 further has one or more computer-readable media, such as the memory 108. The memory 108 includes any quantity of media associated with or accessible by the computing device 102. The memory 108 may be internal to the computing device 102 (as shown in FIG. 1), external to the computing device (not shown), or both (not shown). In some examples, the memory 108 includes read-only memory and/or memory wired into an analog computing device.

The memory 108 stores data, such as one or more applications. The applications, when executed by the processor 106, operate to perform functionality on the computing device 102. The applications may communicate with counterpart applications or services such as web services accessible via a network 112. For example, the applications may represent downloaded client-side applications that correspond to server-side services executing in a cloud.

In other examples, the user interface device 110 includes a graphics card for displaying data to the user and receiving data from the user. The user interface device 110 may also include computer-executable instructions (e.g., a driver) for operating the graphics card. Further, the user interface device 110 may include a display (e.g., a touch screen display or natural user interface) and/or computer-executable instructions (e.g., a driver) for operating the display. The user interface device 110 may also include one or more of the following to provide data to the user or receive data from the user: speakers, a sound card, a camera, a microphone, a vibration motor, one or more accelerometers, a BLUETOOTH® brand communication module, global positioning system (GPS) hardware, and a photoreceptive light sensor. For example, the user may input commands or manipulate data by moving the computing device 102 in one or more ways.

The network 112 is implemented by one or more physical network components, such as, but without limitation, routers, switches, network interface cards (NICs), and other network devices. The network 112 may be any type of network for enabling communications with remote computing devices, such as, but not limited to, a local area network (LAN), a subnet, a wide area network (WAN), a wireless (Wi-Fi) network, or any other type of network. In this example, the network 112 is a WAN, such as the Internet. However, in other examples, the network 112 may be a local or private LAN.

In some examples, the system 100 optionally includes a communications interface component 114. The communications interface component 114 includes a network interface card and/or computer-executable instructions (e.g., a driver) for operating the network interface card. Communication between the computing device 102 and other devices may occur using any protocol or mechanism over any wired or wireless connection. In some examples, the communications interface component 114 is operable with short range communication technologies such as by using near-field communication (NFC) tags.

In at least some examples, the communications interface component 114 enables communications between the computing device 102 and other devices, such as but not limited to a set of one or more robotic plant-watering devices 116 for watering plants and/or moving plants in a plant center, a set of one or more sensor devices 118 generating sensor data 119 associated with the plant center and/or a plurality of data sources 120 providing real-time context data 122 associated with the plant center.

The plurality of data sources 120 provides the real-time context data 122 associated with one or more plants in the live plant center, one or more plant displays within the live plant center, one or more areas of the live plant center, the conditions within the live plant center and/or the conditions/weather surrounding the live plant center. A live plant center is any location storing and/or displaying one or more plants. A live plant center may include a plant nursery, a garden center, a plant display, a set of one or more shelves in a store including one or more plants, or any other area or location including a set of plants. The plurality of data sources 120 in some examples includes, without limitation, one or more news feeds, one or more weather feeds and/or access to one or more shipping and receiving databases/records.

A shipping and receiving database/record includes data associated with expected dates and/or times of arrival of a delivery or shipment of plants, inventory of plants to be delivered on a specific date, delays associated with an expected delivery/shipment of plants or any other data associated with orders of plants to be delivered or shipped to the live plant center.

The system 100 may optionally include a data storage device 124 for storing data, such as, but not limited to a set of per-plant maintenance rules 126. The set of per-plant maintenance rules 126 includes one or more rules for maintaining a specific plant or a type of plant. For example, the per-plant maintenance rules 126 may include a rule specifying that succulent plants are to be watered one time per week until the soil in each pot/container is thoroughly soaked and water begins to run out through the drain holes.

The data storage device 124 may include one or more different types of data storage devices, such as, for example, one or more rotating disks drives, one or more solid state drives (SSDs), and/or any other type of data storage device. The data storage device 124 in some non-limiting examples includes a redundant array of independent disks (RAID) array. In other examples, the data storage device 124 includes a database.

The data storage device 124 in this example is included within the computing device 102 or associated with the computing device 102. In other examples, the data storage device 124 may be a remote data storage accessed by the computing device via the network 112, such as a remote data storage device, a data storage in a remote data center, or a cloud storage.

The memory 108 in some examples stores one or more computer-executable components. Exemplary components include a plant maintenance component 128 that is executed by the processor 106 of the computing device 102. The plant maintenance component 128 generates dynamic per-plant watering schedule 130 for a selected plant or plant type, set of maintenance instructions 132 for watering one or more plants on a selected plant display and/or a set of per-plant watering instructions 132.

A plant display is a device for displaying one or more plants. A plant display may include one or more shelves, cabinets, counters, boxes, trays, etc. In other examples, a plant display may include a rolling plant display, a rolling dolly or movable platform for displaying and/or transporting plants. The plants on a display may be any size plants, from small succulents to large trees.

The per-plant watering schedule 130 includes scheduled dates and/or times for watering a plant or type of plant. The schedule specifies when to water, frequency of watering, etc.

The per-plant watering instructions include directions for quantity of water to apply to each plant based on the plant type, volume of the plant pot/container, temperature, humidity, plant condition, etc. The instructions for watering each plant or plant type may be customized to a specific plant or plants of a given type on a specific display or display area. In other words, plants of the same type may have different watering instructions or different watering schedule based on where the plant is located. For example, a plant located outside in hot weather may require more frequent watering than a plant located under a tarp or other shaded area.

In some examples, the watering instructions may include a next scheduled watering time, a quantity of water and/or a duration of watering for the next scheduled watering time of a given plant or plants of a selected plant type in a selected display or display area.

The update(s) 134 are performed in response to changes in context data and/or changes in sensor data indicating an increase or decrease in watering of plant(s) on a one or more plant displays. For example, a change in sensor data and/or context data may indicate changes in status/condition of one or more plants on a display, a change in weather/temperature, a change in delivery/shipping schedule, etc. If real-time context data and sensor data indicates a change in conditions associated with a plant, the plant maintenance component 128 performs one or more update(s) 134 of a pre-existing set of watering schedule or watering instructions to reflect the changing conditions. For example, if temperatures increase, the watering schedule frequency is increased, and/or the quantity of water applied to each plant is increased to compensate for the higher temperatures.

In this non-limiting example, the plant maintenance component is executed on the computing device 102. In other example, the plant maintenance component 128 may be implemented on one or more of the robotic plant-watering devices and/or one or more smart display devices. A smart display device is a display having a control device including a processor capable of performing analytics and communicating with the robotic plant-watering device.

Thus, the analytics performed by the plant maintenance component may be performed entirely on a robotic plant-watering device or the analytics may be performed by two or more robotic plant-watering devices sharing the processor load as well as one or more smart display devices. In these example, a load balancer may be utilized to divide the processing load among the robotic plant-watering devices and/or one or more smart display devices.

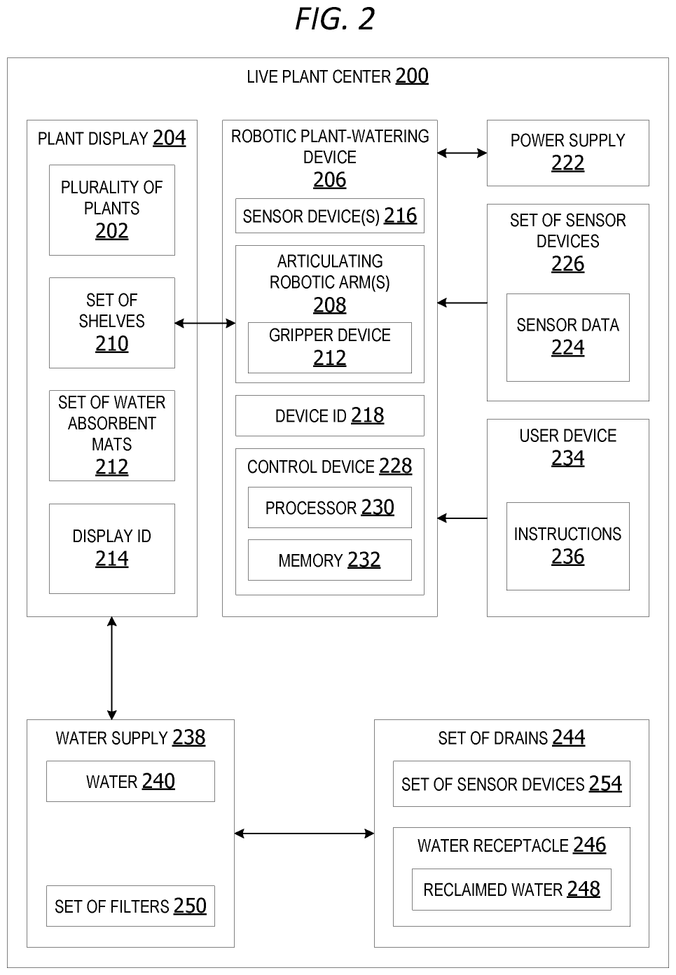

FIG. 2 is an exemplary block diagram illustrating a live plant center 200 including at least one robotic plant-watering device 206. The live plant center 200 in this example is a plant nursery, a garden center or other retail location storing a plurality of plants 202 on a set of one or more plant displays, such as, but not limited to, the plant display 204. The plant display 204 includes one or more plant display devices. The plant display 204 may be a smart display device including a processor, memory and/or a communications interface device enabling the smart display device to send and receive data via the network.

The robotic plant-watering device 206 includes one or more articulating robotic arm(s) 208 having a gripper device 212 for moving/repositioning plants on one or more plant displays or plant display areas.

The robotic plant-watering device 206 selects a specific plant display based on a display identifier (ID) 214 on each plant display. The display ID 214 is an identifier distinguishing the plant display 204 from other plant displays. The display ID 214 may be implemented as a universal product code (UPC), a matrix barcode, a quick response (QR) code, a radio frequency identification (RFID) tag, a label, or any other type of barcode or identifier. The robotic plant-watering device 206 in this example scans the display ID 214 using one or more sensor device(s) 216. The sensor device(s) 216 may include, without limitation, a barcode reader, a camera for capturing an image of the display ID 214, an RFID tag reader, a QR code reader, or any other type of device for reading the display ID 214. In other examples, the one or more sensor device(s) 216 may include an image capture device (camera), motion sensor, pressure sensor, weight sensor, heat sensor, temperature sensor, hygrometer, proximity sensor, light sensor, or any other type of sensor device.

The robotic plant-watering device 206 may optionally include a device ID 218 identifying the robotic plant-watering device 206. A user or other smart device scans/reads the device ID 218 to identify a selected robotic plant-watering device if there are two or more robotic plant-watering devices in the live plant center 200.

The system may also output a notification to the robotic plant-watering device to remove one or more plants which have been incorrectly placed onto a selected plant display. For example, if a plant display contains all bushes, and a user mistakenly misplaces an annual flowering plant on the plant display, that succulent plant would be subjected to non-optimal watering schedules suited to bushes rather than annual flowers. The notice may include a display ID identifying the plant display and a plant ID identifying the annual plant to be removed from the plant display and returned to its correct location elsewhere in the plant center.

The system may also output instructions to the robotic plant-watering device to relocate/reposition items on a cart to improve overall appearance/display value of the item. For example, if plants are arranged so that all red flowering plants are grouped together, and white flowering plants are grouped together on another display, the system outputs a notification requesting removal/relocation of any white flowering plants mistakenly mixed in with the red flowering plants to improve appearance of the plants and display value of the displays, as well as ease of locating desired plants.

The plant display 204 includes a set of one or more water absorbent mats 220 in some examples. The plurality of plants 202 on the plant display 204 are arranged on one or more shelves. Each shelf in this non-limiting example includes a water absorbent mat laying on a surface of the shelf. One or more plants are placed on top the mat on a shelf. When the plants on the display are watered, the water is sprayed onto the water absorbent mat. The mat holds at least some of the water, which is absorbed through one or more holes in the plant pot/container. Plants in containers sitting/resting or otherwise in contact with the wet mat absorbs some of the water from the mat into the container through one or more holes/water-permeable portion of the containers. In some examples, water in the mat flows into a plant pot or other plant storage container via one or more holes in a bottom portion or underside of the pot/storage container in contact with the mat. In this manner, the plants are watered via the set of water absorbent mats rather than spraying the water across the top of the plants or pouring the water into the pot/container for each plant.

The robotic plant-watering device 206 may optionally be powered or recharged via a power supply 222. The power supply may be implemented as a set of batteries, an electrical power source, one or more solar panels attached to the robotic plant-watering device 206, or any other power source.

The robotic plant-watering device 206 in this example generates sensor data via one or more sensor devices on the robotic plant-watering device 206. In other examples, the robotic plant-watering device 206 may receive sensor data 224 from a set of one or more sensor devices 226 within the live plant center 200. The sensors in the set of sensor devices 226 may be located on plant displays, carts, walls, ceilings, on point-of-sale (POS) devices or located anywhere else within the live plant center. A plant display may include a hanging plant display, a table or other fixture on which plants are resting, a bin, a shelf, a cabinet, a counter, or any other display.

In this example, the robotic plant-watering device 206 includes a control device 228 including a memory 232 communicatively coupled to a processor 230. The processor optionally executes a plant maintenance component for generating maintenance instructions, such as, but not limited to, the plant maintenance component 128 in FIG. 1. The instructions may identify a plant display for watering, identify a date and/or time for watering plants on the plant display and/or instructions to move the plant display to a different location. For example, if sensor data or context data indicates a temperature decrease (possibility of frost), the instructions may indicate the robotic plant-watering device 206 should move the plant display 204 inside a protected/sheltered/heated area or be placed below a plastic cover/roof or other protection from the weather.

In other examples, the robotic plant-watering device 206 receives the instructions 236 from a remote computing device, such as a user device 234. The user device is a computing device for generating a set of maintenance instructions, such as, but not limited to, the computing device 102 in FIG. 1.

A user may utilize the user device to manually trigger watering of a plant or type of plant by the robotic plant-watering device if the robotic plant-watering device fails to begin watering automatically or the user determines the plant requires supplemental watering outside scheduled watering. For example, if a plant falls over and has to be repotted with new soil, the plant may require unscheduled watering.

In other examples, the user device may be utilized by the user to manually trigger a robotic plant-watering device to grasp/grab a selected plant and move it to a different location for markdown pricing, to stock a new display, or any other reason. In other words, a user may manually enter a display ID and prompt the robotic plant-watering device to move one or more selected plants from the first selected display to a different, second selected display. For example, if a new shipment of plants is expected, the system may rearrange plants on the displays to make room for the newly arriving plants on one or more displays. The robotic plant-watering device 206 may utilize a set of navigation instructions to guide the robotic plant-watering device 206 from a first assigned location of a plant to the location newly assigned location of the plant. The assigned location is the designated or correct location for a plant. For example, if all fruit trees are located within a roped off area in a patio area, any fruit tree outside that designated space is in an unassigned locations. If the robotic plant-watering device detects a fruit tree mixed in with shade trees, the robotic plant-watering device may utilize one or more gripper devices to pull or carry the tree back to its assigned location with the other fruit trees.

A water supply 238 is provided to refill a water tank on the plant-watering device with water 240. The robotic plant-watering device docks with the water supply to autonomously refill the tank.

The live plant center 200 in at least some examples includes a set of drains 244 for collecting/reclaiming water run-off from the plant display 204 during watering. The set of drains may be located in the floor or in a bottom portion of the sprinkler device or any other device for capturing water run-off from the plant displays. The reclaimed water 248 acts as an additional water supply for the robotic plant-watering device. The robotic plant-watering device re-uses the reclaimed water to water plants on the plant display 204 and/or other plant displays.

The reclaimed water 248 is collected in a water reclamation receptacle. The water receptacle 246 may include one or more water storage containers or reservoirs for holding reclaimed/captured water. The water receptacle may include a set of one or more sensor devices 254 for measuring/determining quality of the reclaimed water, identify any impurities or additives in the water, temperature of the water, etc. For example, if fertilizer is added to the water, the sensor device detects the concentration of the fertilizer in parts per million (PPM). The sensor may also be utilized to identify hard water, water which is too hot or too cold for use on the plants, etc. The sensor data generated by the set of sensor devices 254 may optionally be sent to the user device 234, a data storage device, a cloud server/cloud storage, and/or the robotic plant-watering device 206 for storage and/or additional analysis.

In at least some examples, the water 240 may be filtered through a set of filters 250 to remove particulate matter or other impurities/additives from the reclaimed water 248 prior to storage or prior to use.

In still other examples, the set of sensor devices 254 may also include water quality sensors for detecting impurities, additives, particulate matter and other contents of the water 240 prior to spraying on the plant display(s). If the sensor data indicates the water is too acidic or otherwise contains undesirable additives, an instruction may be generated instructing a user to add a neutralizer to the water 240 to counteract the effects of the undesired elements.

The water reclamation system in this example reclaims water run-off from the plant displays during watering. In other examples, the water reclamation system catches rain water and/or utilizes rainwater captured in rain barrels or other water receptacles.

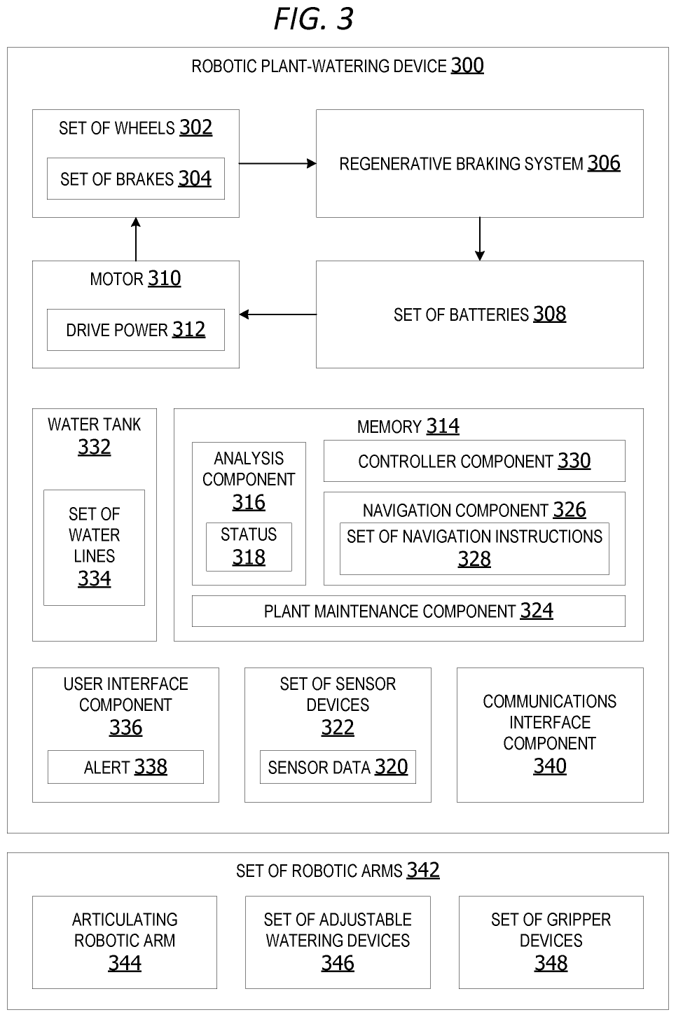

FIG. 3 is an exemplary block diagram illustrating a robotic plant-watering device 300. The robotic plant-watering device 300 includes a set of one or more wheels 302 having a set of one or more brakes 304. When a brake is applied to one of the wheels, a regenerative braking system 306 converts kinetic energy into power to recharge one or more batteries in a set of batteries 308. The set of batteries 308 may be utilized to power a motor 310. The motor may be implemented as an electric motor or any other type of motor generating drive power 312 to turn one or more of the wheels.

In at least some example, a memory 314 includes an analysis component 316 implemented by at least one processor on the robotic plant-watering device 300. The analysis component analyzes sensor data 320 generated by a set of sensor devices 322 associated with at least a portion of a live plant center with real-time context data associated with a plurality of plant using a set of plant maintenance rules. The analysis component 316 generates a status 318 of one or more plants on one or more plant displays based on a result of the analysis. A plant maintenance component 324 utilizes the status 318 to identify a selected plant for watering next.

A navigation component 326 generates a set of navigation instructions 328 for moving the robotic plant-watering device to navigate around the plant center, move a selected plant to a new location or water a selected plant autonomously. The navigation component 326 optionally also generates instructions to move the robotic plant-watering device to a water-refill station to refill the water tank 332.

The water tank 332 may optionally include a set of one or more water lines 334 connecting the water tank to the set of robotic arms 342. In this example, a water line in the set of water lines runs through a hollow central cavity within the articulating robotic arm 344. The water line carries water from the water tank 332 to an adjustable watering device on an end of the end of the articulating robotic arm.

Water sprays out of one or more holes within the adjustable watering device. The robotic plant-watering device moves the articulating robotic arm 344 through a set of motions to spray water evenly across a desired watering zone on the plant. The watering zone may be the soil within the pot/container, an absorbent mat under the plant, or any other portion of a plant or plant container.

In other example, the robotic plant-watering device 300 may move a plant to a different location after watering. For example, if the plants will soon be out-of-season or the sensor data indicates the plants are slightly yellowing/wilted, the plants may be moved to a clearance area or other backroom area for later disposition.

A controller component 330 on the robotic plant-watering device 300 in other examples triggers activation of a set of robotic arms 342 for moving/manipulating plants on a display. The set of robotic arms 342 includes at least one articulating robotic arm 344. The articulating robotic arm 344 includes a set of one or more adjustable watering devices 346 which optionally attach to an end of the articulating robotic arm 344. The articulating robotic arm 344 may also include a set of one or more gripper devices 348.

The robotic plant-watering device optionally includes a user interface component 336 for outputting data to a user and/or receiving data from a user. For example, the user interface component 336 may output an alert 338 if the robotic plant-watering device is malfunctioning or requires maintenance.

The robotic plant-watering device 300 in this example includes a communications interface component 340. The communications interface component 340 enables the robotic plant-watering device to send and receive data via a network, such as, but not limited to, the network 112 in FIG. 1.

The robotic plant-watering device 300 in another example includes an on-board data storage device for storing sensor data, watering schedules, status data, historical data, barcode data, or any other data utilized by the robotic plant-watering device 300 for maintaining plants on one or more plant displays.

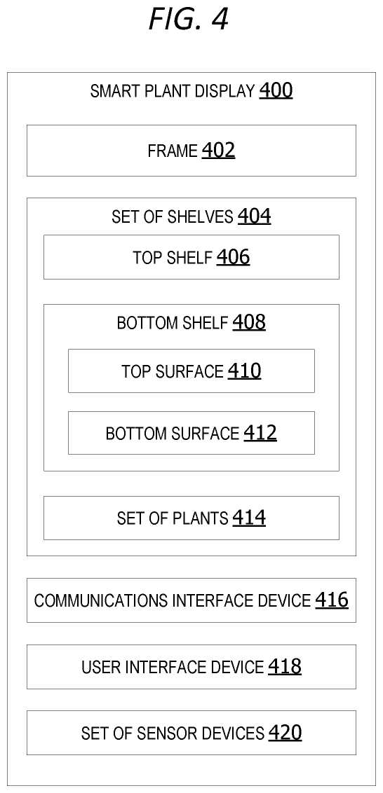

FIG. 4 is an exemplary block diagram illustrating a smart plant display 400 storing a set of plants scheduled for watering. The smart plant display 400 includes a frame 402 including a set of one or more shelves 404. The set of shelves in this non-limiting example includes a top shelf 406 and a bottom shelf 408. In other examples, the set of shelves may include a single shelf, as well as three or more shelves.

The bottom shelf 408 may include a top surface 410 and a bottom surface 412. An absorbent mat may be placed on the top surface 410. One or more plants may be placed on top of the absorbent mat.

A set of wheels (not shown) may optionally be attached to the bottom surface 412 of the bottom shelf 408. The bottom shelf may also be referred to as the lowest shelf or a bottom member of the display. The smart plant display 400 may optionally include a set of one or more sensor devices 420 for generating sensor data associated with plant(s) on the smart plant display 400 and/or conditions surrounding the smart plant display 400 and a communications interface device 416 enabling the display to communicate with one or more other devices via a network, such as, but not limited to, robotic plant-watering devices, user device and/or a cloud server.

The smart plant display 400 may include a user interface device 418 for outputting data to a user and/or receiving data from the user. The user interface device 418 may include a speaker, a display screen/touch screen, a voice recognition system, a set of lights, a printer device, or any other type of user interface.

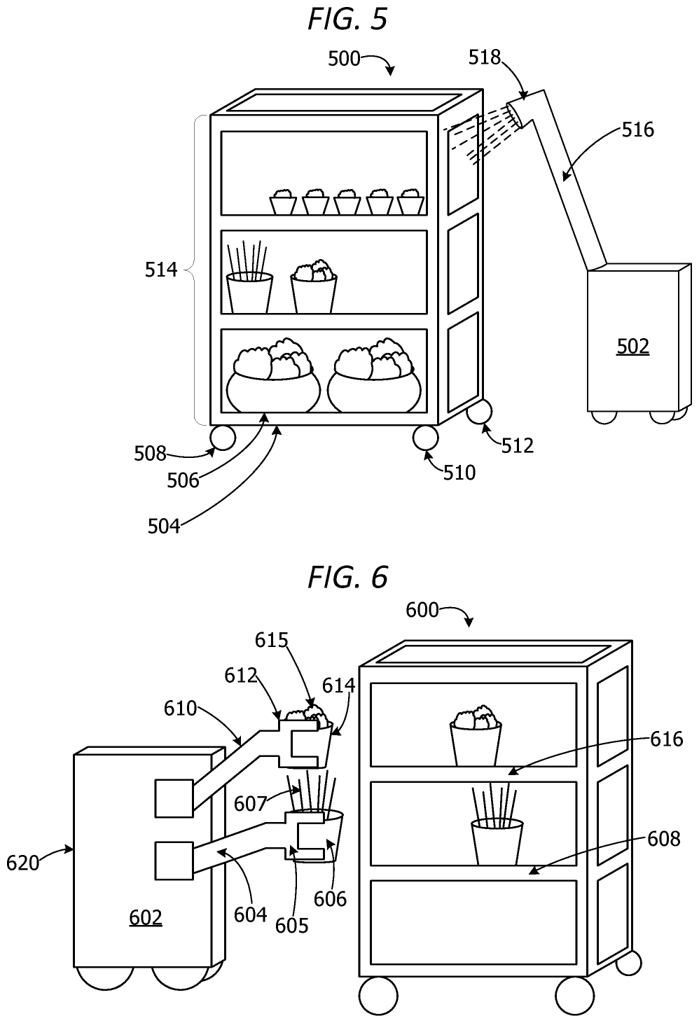

FIG. 5 is an exemplary block diagram illustrating a robotic plant-watering device 502 watering plants on a plant display 500. The plant display 500 in this example includes a set of shelves 514 for displaying one or more live plants. In this example, the set of shelves 514 includes four shelves. In other examples, the plant display 500 may include a single shelf, two shelves, as well as three or more shelves. The plant display in this example contains small potted plants. In other examples, however, the display may hold larger items such as fruit trees, shade trees, or other larger plant types.

The robotic plant-watering device 502 autonomously waters plants on the plant display 500 via at least one robotic arm 516 including a watering device 518 on a portion of the robotic arm 516. The robotic arm 516 in this example is a telescoping arm which extends to reach the highest shelf and retracts to reach plants on the middle and lower shelves. The watering device 518 sprays water extracted from a water tank on the robotic plant-watering device 502.

FIG. 6 is an exemplary block diagram illustrating a robotic plant-watering device 602 utilizing a set of gripper devices to re-arrange plants on a plant display 600. The robotic plant-watering device utilizes a first gripper device 605 attached to a first robotic arm 604 to grasp a container 606 partially enclosing a first plant 607. The robotic plant-watering device extends the robotic arm 604 to place the plant 607 onto a middle shelf 608 of the plant display 600 in this example.

The robotic plant-watering device in this example include a second robotic arm 610 having a second gripper device 612 attached to an end of the robotic arm 610. The robotic plant-watering device utilizes the gripper device 612 to grasp a container 614 partially enclosing a plant 615. The robotic plant-watering device removes the plant 615 from the upper shelf 616 for relocation to another display table or display area.

The robotic arms 604 and 610 are extendable (telescoping) arms. In other examples, the robotic arms may be folding arms which fold to decrease length and unfold to increase length. In still other examples, the robotic arms may be implemented as retractable arms which retract into a recess or other compartment within the main body 620 of the robotic plant-watering device.

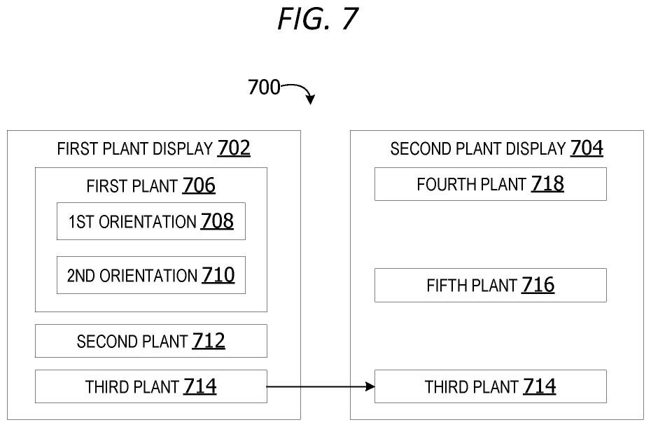

FIG. 7 is an exemplary block diagram illustrating a set of plant displays 700 including a set of plants re-arranged by a robotic plant-watering device. The first plant display 702 includes a first plant 706 which is repositioned by the robotic plant-watering device from a first orientation 708 to a second orientation 710 without removing the plant from the first plant display. For example, if the first plant 706 falls onto its side, the robotic plant-watering device moves the plant from its sideways orientation into a correct, upright orientation. In another example, if a flowering plant is oriented such that the bloom is positioned away from users viewing the plant, the robotic plant-watering device turns the plant such that the new orientation faces the bloom towards viewers.

In this example, the first plant display contains the first plant 706, a second plant 712 and a third plant 714. The second plant display contains a fourth plant 718 and a fifth plant 716. The robotic plant-watering device in this example moves the third plant from the first plant display 702 onto the second plant display 704.

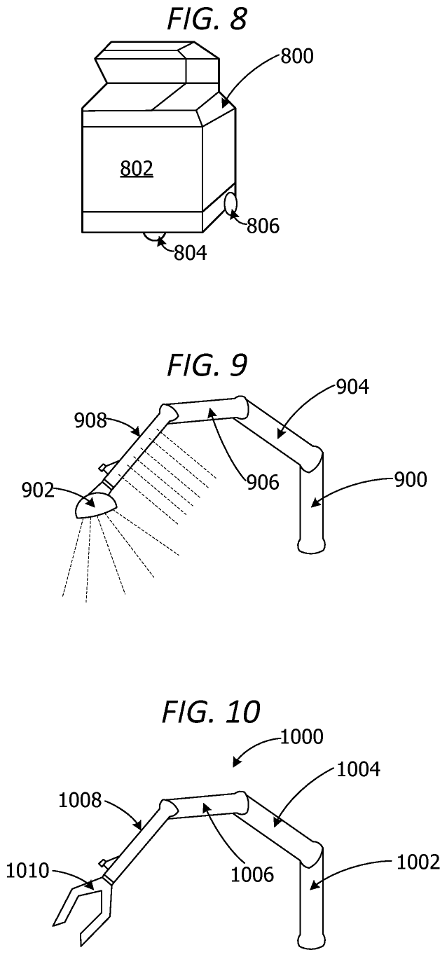

FIG. 8 is an exemplary block diagram illustrating a robotic plant-watering device 800. The robotic plant-watering device 800 in this example includes a set of robotic arms retracted into a main body 802 of the robotic plant-watering device. The robotic plant-watering device includes a set of one or more wheels. The set of wheels in this example includes wheel 804 and wheel 806.

FIG. 9 is an exemplary block diagram illustrating an articulating robotic arm 900 including a set of adjustable watering devices. The set of watering devices are adjustable to control the flow of water from the watering device and/or adjustable to control the direction of water flowing out of the watering device.

The robotic arm 900 in this example is a segmented arm. Each segment enables the arm to bend, fold and/or rotate. A first segment 904 connects to a second segment 906. The second segment 906 connects to a first watering apparatus 908 including a plurality of sprinkler holes for spraying water along a length of the watering apparatus 908. The second watering apparatus 902 is a sprinkler head device attached to an end of the robotic arm 900 for spraying water onto a plant, into a plant container or onto an absorbent mat.

FIG. 10 is an exemplary block diagram illustrating an articulating robotic arm 1000 including a gripper device. The robotic arm 1000 in this non-limiting example includes a base member 1002 connected to a first segment 1004. The first segment is connected to a second segment 1006. The second segment 1006 connects to a third segment 1008. The third segment connects to a gripper device 1010. The gripper device is capable of grabbing or grasping a portion of a plant pot or other container to move or reposition the plant.

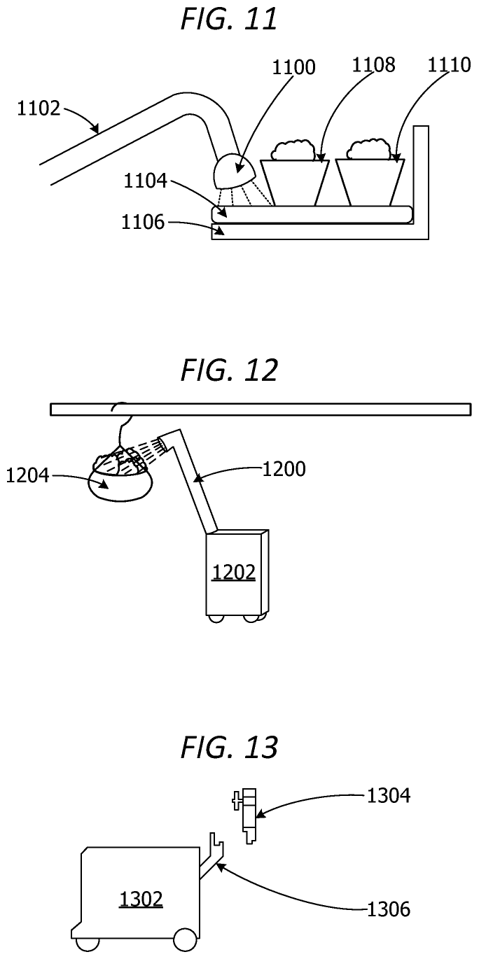

FIG. 11 is an exemplary block diagram illustrating an adjustable watering apparatus 1100. The watering apparatus 1100 connects to a robotic arm of a robotic plant-watering device. In this example, the robotic plant-watering device sprays water onto an absorbent mat 1104 placed on a shelf 1106. The plants 1108 and 1110 resting on the mat 1104 absorb the water through a set of holes in the bottom of the containers.

FIG. 12 is an exemplary block diagram illustrating a telescoping robotic arm 1200 for watering hanging plants. In this example, the robotic plant-watering device 1202 utilizes the robotic arm 1200 to water a hanging plant 1204.

FIG. 13 is an exemplary block diagram illustrating a robotic plant-watering device 1302 connecting to a water refill docking device. In this example, a connection 1306 on the robotic plant-watering device 1302 autonomously attaches to a water refill docking device 1304. Water flows from a water source into a water tank on the robotic plant-watering device via the water refill docking device. The water source may include a public water supply, reclaimed water, a rainwater reservoir or any other water source.

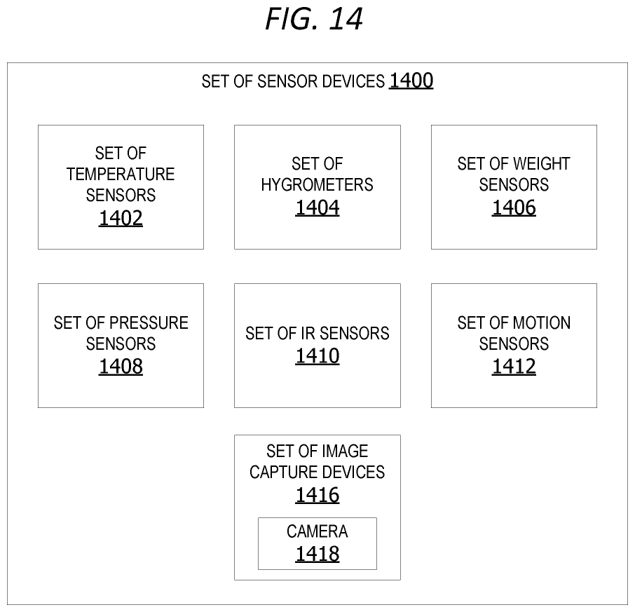

FIG. 14 is an exemplary block diagram illustrating a set of sensor devices 1400. The set of sensor devices 1400 in some example includes sensor devices on a robotic plant-watering device. In other examples, set of sensor devices includes sensor devices on a plant display or a sensor device within the plant center. The set of sensor devices 1400 in this example includes a set of one or more temperature sensors 1402 such as a thermometer, a set of one or more hygrometers 1404 for measuring humidity, a set of one or more weight sensors 1406, a set of one or more pressure sensors 1408, a set of one or more infrared (IR) sensors 1410, a set of one or more motion sensors 1412, a set of one or more scanner devices 1414, and/or a set of one or more image capture devices 1416, such as, but not limited to, a camera 1418. The set of scanner devices 1414 may include a barcode scanner, a QR code reader, an RFID tag reader, or any other type of scanner. A barcode scanner may include a UPC code reader, a matrix barcode reader, etc.

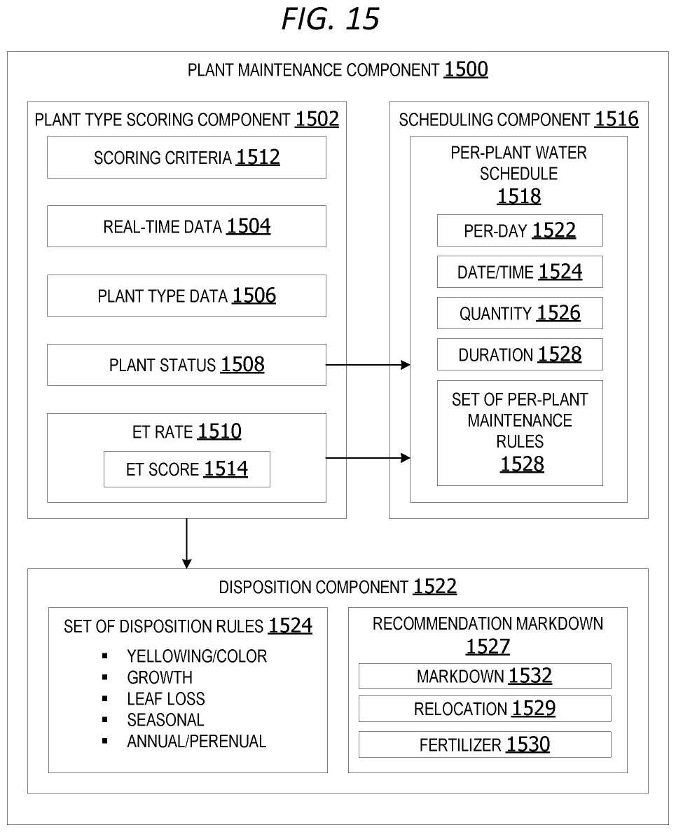

FIG. 15 is an exemplary block diagram illustrating a plant maintenance component 1500. A plant type scoring component 1502 analyzes real-time data 1504 with plant type data 1506 and/or plant status data 1508 to generate an evapotranspiration (ET) rate for a given plant or a given plant type. The plant type data 1506 is data associated with a type of plant. For example, if the plant type is a rose bush, the plant type data 1506 includes data associated with rose bushes, such as, but without limitation, number of hours of sun per day, water saturation/moisture levels, etc.

The plant type scoring component 1502 generates an ET score 1514 for the selected plant and/or the plant type associated with the selected plant based on the ET rate 1510 and a set of scoring criteria 1512. The scoring criteria includes rules for scoring each plant type based on the ET rate.

A scheduling component 1516 generates a per-plant water schedule 1518 for a selected plant or the selected plant type based on the ET score 1514 and a set of per-plant maintenance rules 1520 for that type of plant. The per-plant water schedule is customized based on weather conditions, ambient temperature and humidity in the plant center, the plant type data, etc. The per-plant water schedule 1518 may include how many times to water the plant per-day (daily frequency of watering), a date and/or time for the next schedule watering, a quantity 1526 of water to be applied to the selected plant or to plants of the same selected plant type, and/or duration 1528 for watering. The duration 1528 refers to length of time to spray water on the plant during watering.

A disposition component 1522 generates a recommendation 1527 for disposing of a plant based on the plant's condition based on a set of disposition rules 1524. The recommendation 1527 may include a recommendation to markdown 1532 one or more plants or plant types (reduce price). A markdown may occur when a plant type is out-of-season or almost out-of-season.

A recommendation of relocation 1529 of one or more plants or types of plants from a selected display or display area may be made when context data indicates a one or more plants should be moved to a different location. For example, a plant may be moved to a location with better light, more shade or for better coordination with other plants/plant colors. One or more plants may also be moved to a markdown/clearance area.

A recommendation 1527 may also include a recommendation to add fertilizer 1530 or other additives to the plant or water sprayed on the plant. A recommendation may be made based on a condition of a plant, such as yellowing, change in color, new growth, lack of new growth, wilting, leaf loss, etc. The recommendation may also be made based on weather and season and whether the plant is an annual or perennial.

FIG. 16 is an exemplary block diagram illustrating a cloud server 1600 including an analysis component 1602. The cloud server 1600 in this example implements the analysis component 1602. The analysis component includes an artificial intelligence (AI) with machine learning (ML) permitting the system to analyze real-time data using historical data and training data using pattern analysis to improve watering schedules and plant maintenance/plant quality.

The analysis component performs analytics in this example remotely from the robotic plant-watering device to reduce resource usage on the robotic plant-watering device. The cloud server maintains network connectivity with devices such as the robotic plant-watering device, sensor devices and/or smart plant display devices via the network.

In this example, the analysis component 1602 generates a status update 1604 for a plant based on results of analysis of sensor data and context data. The status update 1604 may include a plant ID 1606, a descriptor 1608 describing the condition and/or appearance of the plant, and/or a location 1610 of the plant.

The analysis component may optionally generate a set of watering instructions 1612 for a plant or plant type. The analysis component in other examples generates an update 1614 for a pre-existing set of watering instructions for a plant or plant type. The analysis component may also perform analysis of real-time data to generate the disposition instructions 1616 for a plant or plant type on the remote cloud server. The disposition instructions may include markdown instructions 1618 to reduce price of a plant/plant type. The disposition instructions include relocation instructions 1620 to remove a plant or plant type to a different location or reposition a plant in its current location.

In still other non-limiting examples, the cloud server performs analytics on real-time plant data to generate the ET rate 1622 and/or the ET score 1624. The cloud server 1600 transmits the status update 1604, set of watering instructions 1612, disposition instructions 1616, ET rate 1622 and/or ET score 1624 to the robotic plant-watering device via a network.

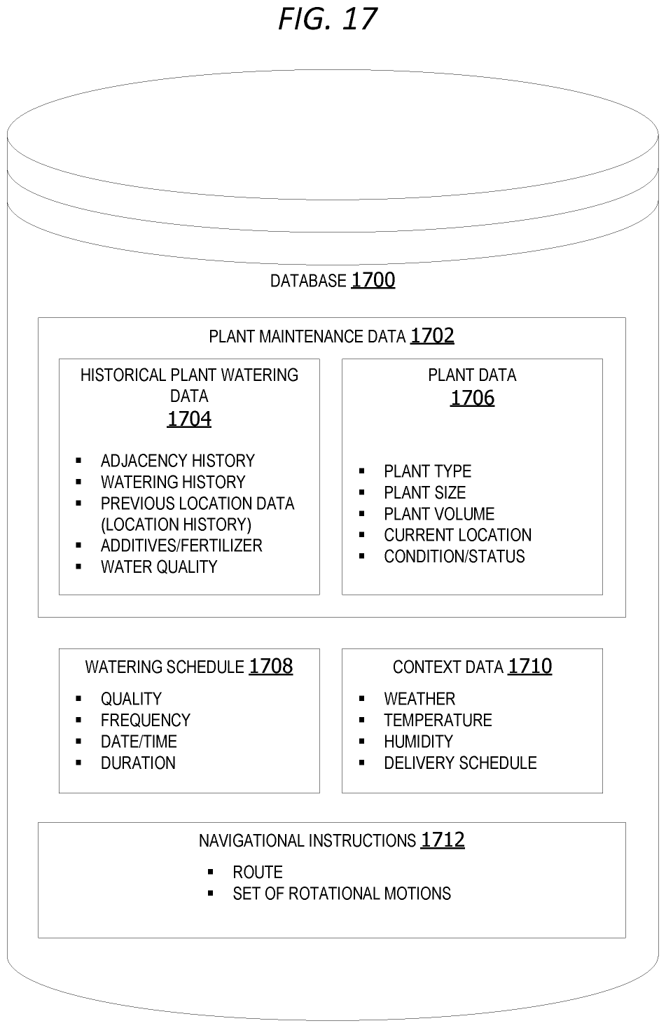

FIG. 17 is an exemplary block diagram illustrating a database 1700 for storing plant maintenance data 1702. The plant maintenance data 1702 may include historical plant watering data 1704 and/or plant data 1706. The historical plant watering data may include adjacency history detailing other plants placed near or adjacent to the plant/plant type on a plant display. The historical plant watering data may include watering history, previous location data, additives/fertilizer added to water and/or water quality of water sprayed on the plants. Plant data 1706 may include plant type, plant size, plant volume (volume of pot/container, current location of plant in plant center, and/or condition or status of the plant.

Watering schedule 1708 includes details for a next scheduled watering of a plant/plant type or plants on one or more plant displays. The watering schedule may specify quantity of water to be sprayed, frequency of watering per day, frequency of watering per-week, frequency of watering per-month, date and/or time of the next scheduled watering, and/or duration of watering time.

Context data 1710 includes data describing current weather, temperature and/or humidity. The context data may also include delivery/shipment schedules detailing when new plants are scheduled for delivery to the plant center and/or the types of plants scheduled for delivery.

Navigational instructions 1712 optionally include one or more routes (directions) between a plant current location and a new location for the plant. The route may also include directions identifying a route/path from the robotic plant-watering device's current location to a water refill docking device. The navigational instructions 1712 may also include a set of rotational motions for moving one or more robotic arms on the robotic plant-watering device. The rotational motions of the arm(s) may include forward motions, backward motions, side-to-side movement, up movements, down movements and/or rotational (spinning) movements to assist with equal spreading of water across a plant, a portion of the plant, and/or an absorbent mat.

FIG. 18 is an exemplary flow chart illustrating operation of the computing device to generate instructions for a robotic plant-watering device. The process shown in FIG. 18 may be performed by a computing device, such as the robotic plant-watering device in FIG. 1.

The process begins by analyzing sensor data and context data using status criteria at 1802. The robotic plant-watering device generates a status update for one or more plants at 1804. The robotic plant-watering device determines if a change in a watering schedule is needed at 1806. If yes, the robotic plant-watering device updates watering instructions for the plant(s) at 1808. The robotic plant-watering device generates navigational instructions to the location of the plant(s) at 1810. The robotic plant-watering device triggers an arm to release water onto the plant(s) at 1812. The robotic plant-watering device updates historical watering data for plant(s) at 1814. The process terminates thereafter.

While the operations illustrated in FIG. 18 are performed by a computing device, aspects of the disclosure contemplate performance of the operations by other entities. For example, a cloud service may perform one or more of the operations shown in FIG. 18.

FIG. 19 is an exemplary flow chart illustrating operation of the computing device to autonomously water plants. The process shown in FIG. 19 may be performed by a computing device, such as the robotic plant-watering device in FIG. 1.

The process begins by identifying a plant scheduled for watering at 1902. The robotic plant-watering device positions a robotic arm with a watering apparatus within watering range of the plant at 1904. The robotic plant-watering device sprays water onto the plant, into the plant's container or on a mat under the plant at 1906. The robotic plant-watering device determines if watering is complete at 1908. If yes, the robotic plant-watering device determines if there is a next plant scheduled for watering at 1910. If yes, the robotic plant-watering device iteratively performs operations 1902 through 1910 until there are no plants requiring watering at 1910. The process terminates thereafter.

While the operations illustrated in FIG. 19 are performed by a computing device, aspects of the disclosure contemplate performance of the operations by other entities. For example, a cloud service may perform one or more of the operations shown in FIG. 19.

FIG. 20 is an exemplary flow chart illustrating operation of the computing device to autonomously refill a water tank on a robotic plant-watering device. The process shown in FIG. 20 may be performed by a computing device, such as the robotic plant-watering device in FIG. 1.

The process begins by analyzing sensor data associated with a water tank on the robotic plant-watering device at 2002. The robotic plant-watering device determines if the water level is below a minimum threshold at 2004. If yes, the robotic plant-watering device navigates to a water supply at 2006. The robotic plant-watering device connects to a water refill docking apparatus at 2008. The robotic plant-watering device adds water to the tank at 2010. The robotic plant-watering device determines if the tank is full at 2012. If yes, the robotic plant-watering device disconnects from the water refill docking apparatus at 2014. The process terminates thereafter.

While the operations illustrated in FIG. 20 are performed by a computing device, aspects of the disclosure contemplate performance of the operations by other entities. For example, a cloud service may perform one or more of the operations shown in FIG. 20.

### Additional Examples

In some examples, the system provides a robot that brings water to one or more plants. The robot autonomously makes determinations about frequency and quantity of water. The robot is also capable of moving plants to different locations/removing plants from display shelves.

In an example scenario, a robotic plant-watering device waters plants at their display locations. The robotic plant-watering device includes a water tank and a robotic arm. The arm is able to extend in various directions and to various lengths to water various types of plants, including, without limitation, pants in hanging baskets, potted plants, etc. The robotic plant-watering device autonomously docks with a water source and/or a power source in order to stay adequately powered and carrying a sufficient quantity of water.

The robotic plant-watering device includes a removable and interchangeable watering apparatus which attaches to one of the robotic arms. The robotic plant-watering device may also include a gripper for moving plants or other items around. The gripper in other examples pulls a plastic sheet or tarp over one or more plants to cover the plants if a frost is expected. The gripper may also be used by the robotic plant-watering device to remove a sheet or other covering from one or more plants after danger of frost is over.

In some examples, the robotic plant-watering device includes a camera for use in identifying the plants, as well as navigating around objects within the plant center. The camera images are analyzed to determine the condition/state of plants, placement/location of plants and/or identify objects within the plant center.

The data processing in some examples is partially performed on the robotic plant-watering device and partially performed on a cloud server. The cloud server stores data and rules for generating dynamic watering schedules and/or watering instructions. The robotic plant-watering device sends data to the cloud server and receives data (instructions) back from the cloud server in other examples. In one example, the robotic plant-watering device uploads data onto the cloud server once per-day.

In other examples, the robotic plant-watering device may exchange information with the cloud more frequently or less frequently. The instructions received from the cloud server may include modified watering instructions, such as, but not limited to, a cessation of watering due to rain or an increase in watering due to a dry spell/heat wave. The instructions may also include directions to move plants to a markdown area or to actually mark down the plants.

The robotic plant-watering device in another example receives sensor data and analyzes it to determine quality/condition of plants, water sprayed on plants, displays, etc. In one example, if the robotic plant-watering device determines that water passing through robotic plant-watering device contains too much phosphorous, the robotic plant-watering device adds a neutralizer to the water.

In still other examples, the robotic plant-watering device captures data from plant IDs on plants, display IDs on display devices, and other sensor data. The system analyzes the data to determine the number of plants on each display/tables where plants are located on each display/table, and whether a location or orientation of a plant should be adjusted. If a plant is moved to an incorrect location, the robotic plant-watering device autonomously moves the plants/returns the plants to each plants proper location. The plants may be moved based on assigned display locations or other location rules/maintenance rules specifying where plants should be kept. For example, a maintenance rule may specify whether a plant should be kept in a sunny location or a shady location. Another rule may specify which types of plants should be kept together and which plants should be located separately.

Alternatively, or in addition to the other examples described herein, examples include any combination of the following:
a set of navigational instructions generated by the controller component, the set of navigational instructions directing movement of the robotic plant-watering device from an assigned location of a first plant to an assigned location of a second plant;
a plurality of data sources providing the real-time context data associated with the live plant center, the plurality of data sources comprising at least one of a news feed, a weather feed and a shipping and receiving database;
an analysis component implemented on the at least one processor of the computing device analyzes the real-time context data associated with the live plant center, the sensor data generated by the set of sensor devices within the live plant center and historical plant data associated with the plurality of plants using the set of per-plant maintenance rules;
the analysis component generates a status update for at least one plant in the plurality of plants based on a result of the analysis, the status update comprising at least one of a descriptor associated with a condition of the at least one plant, an appearance of the at least one plant and a current location of the at least one plant;
the analysis component generates an updated set of per-plant watering instructions for a selected plant type based on a result of the analysis, the updated set of per-plant watering instructions including a date for a next watering of the selected type of plant, a time for the next watering, a duration of the next watering and a quantity of additives to be added to the quantity of water during the watering of at least one plant of the selected plant type;
the analysis component generates disposition instructions for at least one plant in the plurality of plants based on a result of the analysis, the disposition instructions comprising at least one of an instruction to markdown the at least one plant and an instruction to move the at least one plant to a different location;
wherein the set of sensors comprises at least one of a set of thermometers, a set of hygrometers, a set of pressure sensors, a set of weight sensors, a set of motion sensors, a set of image capture devices and a set of scanner devices;
a water refill docking device on the robotic plant-watering device, wherein the water refill docking device connects to a water source to refill the water tank on condition at least one sensor associated with the water tank indicates a level of water within the water tank is below a threshold minimum water level;
a water absorbent mat associated with a selected plant, wherein the robotic plant-watering device outputs water onto the water absorbent mat, wherein the selected plant absorbs the water from the absorbent mat through a bottom portion of a container associated with the plant;
a set of drains below a set of plants configured to catch water draining off the set of plants;
a set of water reclamation reservoirs for storing reclaimed water captured by the set of drains;
a set of filters associated with the set of water lines in the robotic plant-watering device, wherein the set of filters remove particulates from the reclaimed water in the set of water reclamation reservoirs prior to the robotic plant-watering device re-using the reclaimed water to water at least one plant;
gripping, by a gripper device, a portion of the selected plant or a container associated with a selected plant on condition the robotic plant-watering device receives instructions to move the selected plant from a first location to a second location;
moving the gripper device to the second location;
releasing, by the gripper device, the portion of the selected plant or the container to relocate the selected plant to the second location;
analyzing the sensor data to identify a plant to be relocated from a first location to a second location based on the plant type, a color of the plant or a condition of the plant;
gripping, by a gripper device, a portion of the plant or a container associated with the plant;
moving the gripper device holding the plant to the second location;
releasing the portion of the selected plant or the container to relocate the selected plant to the second location;
moving the at least one robotic arm in a set of motions to evenly spray water across the set of plants, wherein the set of motions includes at least one of a forward motion, a backward motion, an upward motion, a downward motion and a circular motion;
obtaining sensor data associated with quality of water in the water tank associated with the robotic plant-watering device from at least one sensor device associated with the water tank or the set of water lines;
analyzing, by a cloud server, real-time weather data associated with the live plant center to generate an evapotranspiration (ET) rate for the live plant center;
generating an ET score for each plant type in the plurality of plants based on the ET rate and item data for each plant type, the item data comprising watering history, plant state data, plant size, plant volume, adjacency history, location history and weather data;
a set of gripper devices associated with at least one articulated robotic arm in the set of articulated robotic arms configured to grasp a portion of a plant or a portion of a container associated with a plant;
a set of filters associated with the water tank or the set of water lines; and
a data storage device storing item data associated with each plant in the plurality of plants.

At least a portion of the functionality of the various elements in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, and FIG. 17 may be performed by other elements in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, and FIG. 17, or an entity (e.g., processor 106, web service, server, application program, computing device, etc.) not shown in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, and FIG. 17.

In some examples, the operations illustrated in FIG. 18, FIG. 19 and FIG. 20 may be implemented as software instructions encoded on a computer-readable medium, in hardware programmed or designed to perform the operations, or both. For example, aspects of the disclosure may be implemented as a system on a chip or other circuitry including a plurality of interconnected, electrically conductive elements.

While the aspects of the disclosure have been described in terms of various examples with their associated operations, a person skilled in the art would appreciate that a combination of operations from any number of different examples is also within scope of the aspects of the disclosure.

The term “Wi-Fi” as used herein refers, in some examples, to a wireless local area network using high frequency radio signals for the transmission of data. The term “BLUETOOTH®” as used herein refers, in some examples, to a wireless technology standard for exchanging data over short distances using short wavelength radio transmission. The term “cellular” as used herein refers, in some examples, to a wireless communication system using short-range radio stations that, when joined together, enable the transmission of data over a wide geographic area. The term “NFC” as used herein refers, in some examples, to a short-range high frequency wireless communication technology for the exchange of data over short distances.

### Exemplary Operating Environment

Exemplary computer-readable media include flash memory drives, digital versatile discs (DVDs), compact discs (CDs), floppy disks, and tape cassettes. By way of example and not limitation, computer-readable media comprise computer storage media and communication media. Computer storage media include volatile and nonvolatile, removable and non-removable media implemented in any method or technology for storage of information such as computer-readable instructions, data structures, program modules and the like. Computer storage media are tangible and mutually exclusive to communication media. Computer storage media are implemented in hardware and exclude carrier waves and propagated signals. Computer storage media for purposes of this disclosure are not signals per se. Exemplary computer storage media include hard disks, flash drives, and other solid-state memory. In contrast, communication media typically embody computer-readable instructions, data structures, program modules, or the like, in a modulated data signal such as a carrier wave or other transport mechanism and include any information delivery media.

Although described in connection with an exemplary computing system environment, examples of the disclosure are capable of implementation with numerous other general purpose or special purpose computing system environments, configurations, or devices.

Examples of well-known computing systems, environments, and/or configurations that may be suitable for use with aspects of the disclosure include, but are not limited to, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform tasks or implement abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more functionality or less functionality than illustrated and described herein.

In examples involving a general-purpose computer, aspects of the disclosure transform the general-purpose computer into a special-purpose computing device when configured to execute the instructions described herein.

The examples illustrated and described herein as well as examples not specifically described herein but within the scope of aspects of the disclosure constitute exemplary means for autonomously watering plants via a robotic device. For example, the elements illustrated in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, and FIG. 17 such as when encoded to perform the operations illustrated in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, and FIG. 17, constitute exemplary means for analyzing sensor data generated by a set of sensor devices associated with a plurality of plants in a live plant center and real-time context data associated with the live plant center using a set of status criteria; exemplary means for generating an updated status for each type of plant in the plurality of plants based on a result of the analysis; exemplary means for updating a set of watering instructions for a selected plant type; exemplary means for generating a set of navigation instructions for navigating the robotic plant-watering device to a current location of the set of plants of the selected plant type; exemplary means for triggering at least one articulating robotic arm to release a quantity of water onto the selected plant for a duration of time in accordance with the updated set of watering instructions; and exemplary means for updating historical watering data for the selected plant on a data storage to reflect completion of a watering task associated with the selected plant. The quantity of water added to each plant or added to one or more plants is a predetermined or specified quantity of water. In other examples, water is added to each plant until sensor data indicates the soil in each plant pot is sufficiently saturated and/or the soil has reached a desired level of moisture.

Other non-limiting examples provide one or more computer storage devices having a first computer-executable instructions stored thereon for autonomously updating plant watering schedules based on real-time sensor data and/or other context data. When executed by a computer, the computer performs operations including analyzing sensor data, generating per-plant watering schedules, generating navigation instructions for navigating a robotic watering device through a live plant center and/or applying a pre-calculated quantity of water onto one or more selected plants in accordance with a watering schedule.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing an operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

In an exemplary embodiment, one or more of the exemplary embodiments include one or more localized Internet of Things (IoT) devices and controllers. As a result, in an exemplary embodiment, the localized IoT devices and controllers can perform most, if not all, of the computational load and associated monitoring and then later asynchronous uploading of summary data can be performed by a designated one of the IoT devices to a remote server. In this manner, the computational effort of the overall system may be reduced significantly. For example, whenever localized monitoring allows remote transmission, secondary utilization of controllers keeps securing data for other IoT devices and permits periodic asynchronous uploading of the summary data to the remote server. In addition, in an exemplary embodiment, the periodic asynchronous uploading of summary data may include a key kernel index summary of the data as created under nominal conditions. In an exemplary embodiment, the kernel encodes relatively recently acquired intermittent data (“KRI”). As a result, in an exemplary embodiment, KRI includes a continuously utilized near term source of data, but KRI may be discarded depending upon the degree to which such KRI has any value based on local processing and evaluation of such KRI. In an exemplary embodiment, KRI may not even be utilized in any form if it is determined that KRI is transient and may be considered as signal noise. Furthermore, in an exemplary embodiment, the kernel rejects generic data to provide a modified kernel (“KRG”) by filtering incoming raw data using a stochastic filter that thereby provides a predictive model of one or more future states of the system and can thereby filter out data that is not consistent with the modeled future states which may, for example, reflect generic background data. In an exemplary embodiment, KRG incrementally sequences all future undefined cached kernels of data to filter out data that may reflect generic background data. In an exemplary embodiment, KRG further incrementally sequences all future undefined cached kernels having encoded asynchronous data to filter out data that may reflect generic background data.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A system for robotic plant watering based on dynamic context data, the system comprising:
a plurality of plants associated with a plant display in a live plant center:
a set of sensor devices generating sensor data associated with the plurality of plants, the sensor data comprising at least one of temperature data and image data;
a plant maintenance component, implemented on at least one processor associated with a computing device, generates a dynamic per-plant watering schedule based on an analysis of the sensor data and real-time context data associated with the live plant center using a set of per-plant maintenance rules;
a robotic plant-watering device configured to water at least one plant in the plurality of plants in accordance with the dynamic per-plant watering schedule, the robotic plant-watering device comprising:
a water tank configured to store water;
a set of water lines connecting the water tank to a set of articulating robotic arms;
an adjustable watering apparatus removably attached to a first robotic arm in the set of robotic arms, the adjustable watering apparatus configured to release a quantity of water from the water tank onto the at least one plant;
a gripper device removably attached to a second robotic arm in the set of robotic arms, the gripper device configured to grasp a portion of a container associated with the at least one plant;
the gripper device moves the at least one plant from a first location to a second location or repositions the at least one plant from a first orientation to a second orientation;
a control device comprising at least one processor communicatively coupled to a memory;
a controller component, implemented on the at least one processor, triggers release of the quantity of water from the adjustable watering apparatus onto the at least one plant for a watering duration specified in the dynamic per-plant watering schedule.

2. The system of claim 1, further comprising:
navigational instructions generated by the controller component, the navigational instructions directing movement of the robotic plant-watering device from an assigned location of a first plant to an assigned location of a second plant.

3. The system of claim 1, further comprising:
a plurality of data sources providing the real-time context data associated with the live plant center, the plurality of data sources comprising at least one of a news feed, a weather feed and a shipping and receiving database.

4. The system of claim 1, further comprising:
an analysis component implemented on the at least one processor of the computing device analyzes the real-time context data associated with the live plant center, the sensor data generated by the set of sensor devices within the live plant center and historical plant data associated with the plurality of plants using the set of per-plant maintenance rules; and
the analysis component generates a status update for the at least one plant in the plurality of plants based on a result of the analysis, the status update comprising at least one of a descriptor associated with a condition of the at least one plant, an appearance of the at least one plant and a current location of the at least one plant.

5. The system of claim 1, further comprising:
an analysis component implemented on the at least one processor of the computing device analyzes the real-time context data associated with the live plant center, the sensor data generated by the set of sensor devices within the live plant center and historical plant data associated with the plurality of plants using the set of per-plant maintenance rules; and
the analysis component generates an updated set of per-plant watering instructions for a selected plant type based on a result of the analysis, the updated set of per-plant watering instructions including a date for a next watering of the selected plant type, a time for the next watering, a duration of the next watering and a quantity of additives to be added to the quantity of water during the watering of the at least one plant of the selected plant type.

6. The system of claim 1, further comprising:
an analysis component implemented on the at least one processor of the computing device analyzes the real-time context data associated with the live plant center, the sensor data generated by the set of sensor devices within the live plant center and historical plant data associated with the plurality of plants using the set of per-plant maintenance rules; and
the analysis component generates disposition instructions for the at least one plant in the plurality of plants based on a result of the analysis, the disposition instructions comprising at least one of an instruction to markdown the at least one plant and an instruction to move the at least one plant to a different location.

7. The system of claim 1, wherein the set of sensors comprises at least one of a set of thermometers, a set of hygrometers, a set of pressure sensors, a set of weight sensors, a set of motion sensors, a set of image capture devices and a set of scanner devices.

8. The system of claim 1, further comprising:
a water refill docking device on the robotic plant-watering device, wherein the water refill docking device connects to a water source to refill the water tank on condition at least one sensor associated with the water tank indicates a level of water within the water tank is below a threshold minimum water level.

9. The system of claim 1, further comprising:
a water absorbent mat associated with a selected plant, wherein the robotic plant-watering device outputs water onto the water absorbent mat, wherein the selected plant absorbs the water from the absorbent mat through a bottom member of a container at least partially enclosing a plant, wherein the bottom member of the container is in contact with the absorbent mat.

10. The system of claim 1, further comprising:
a set of drains below a set of plants configured to catch water draining off the set of plants;
a water reclamation receptacle for storing reclaimed water captured by the set of drains; and
a set of filters associated with the set of water lines in the robotic plant-watering device, wherein the set of filters remove particulates from the reclaimed water in the water reclamation receptacle prior to the robotic plant-watering device re-using the reclaimed water to water the at least one plant.

11. A computer-implemented method for dynamically watering plants via a robotic plant-watering device, the computer-implemented method comprising:
analyzing sensor data generated by a set of sensor devices associated with a plurality of plants in a live plant center and real-time context data associated with the live plant center using a set of status criteria;
generating an updated status for each type of plant in the plurality of plants based on a result of the analysis;
updating, by a plant maintenance component, a set of watering instructions for a selected plant type, the updated set of watering instructions comprising a next scheduled watering time and an amount of water to be applied to a set of plants of the selected plant type during the next scheduled watering time;
generating, by a navigation system, a set of navigation instructions for navigating the robotic plant-watering device to a current location of the set of plants of the selected plant type;
triggering, by a control device, at least one articulating robotic arm to release a quantity of water onto the selected plant for a duration of time in accordance with the updated set of watering instructions; and
updating, by the plant maintenance component, historical watering data for the selected plant on a data storage to reflect completion of a watering task associated with the selected plant.

12. The computer-implemented method of claim 11, further comprising:
gripping, by a gripper device, a portion of the selected plant or a container associated with a selected plant on condition the robotic plant-watering device receives instructions to move the selected plant from a first location to a second location;
moving the gripper device to the second location; and
releasing, by the gripper device, the portion of the selected plant or the container to relocate the selected plant to the second location.

13. The computer-implemented method of claim 11, further comprising:
analyzing the sensor data to identify a plant to be relocated from a first location to a second location based on a type of plant, a color of the plant or a condition of the plant;
gripping, by a gripper device, a portion of the plant or a container associated with the plant;
moving the gripper device holding the plant to the second location; and
releasing the portion of the selected plant or the container to relocate the selected plant to the second location.

14. The computer-implemented method of claim 11, further comprising:
moving the first robotic arm in a set of motions to evenly spray water across the set of plants, wherein the set of motions includes at least one of a forward motion, a backward motion, an upward motion, a downward motion and a circular motion.

15. The computer-implemented method of claim 11, further comprising:
obtaining the sensor data by at least one sensor device associated with a water tank or a set of water lines associated with the robotic plant-watering device; and
analyzing the sensor data to determine quality of the water prior to spraying the water onto at least one plant.

16. The computer-implemented method of claim 11, further comprising:
analyzing, by a cloud server, real-time weather data associated with the live plant center to generate an evapotranspiration (ET) rate for the live plant center; and
generating an ET score for each plant type in the plurality of plants based on the ET rate and item data for each plant type, the item data comprising watering history, plant state data, plant size, plant volume, adjacency history, location history and weather data.

17. A robotic plant-watering device comprising:
a set of articulated robotic arms connected to a main body;
a set of adjustable water sprinkler devices removably attached to at least one articulated robotic arm in the set of articulated robotic arms configured to release a quantity of water from a water source via a set of apertures on each water sprinkler device;
a set of sensor devices generating sensor data, the set of sensor devices comprising a set of image capture devices and a set of water quality sensors associated with the water source;
a control device comprising a memory communicatively coupled to at least one processor;
a plant maintenance component implemented on the at least one processor analyzes the sensor data using a set of plant maintenance rules to generate a dynamic plant watering schedules for a selected plant in a plurality of plants based on a current status of the selected plant and current ambient conditions within a plant center;
a controller component implemented on the at least one processor moves at least one robotic arm within a proximity of the selected plant; and
the controller component triggers release of the quantity of water specified in the dynamic plant watering schedule for the selected plant onto the selected plant.

18. The robotic plant-watering device of claim 17 further comprising:
a set of gripper devices associated with at least one articulated robotic arm in the set of articulated robotic arms configured to grasp a portion of a plant or a portion of a container associated with a plant.

19. The robotic plant-watering device of claim 17 further comprising:
a set of filters associated with a water tank or a set of water lines.

20. The robotic plant-watering device of claim 17, further comprising:
a data storage device storing item data associated with each plant in the plurality of plants.
